(19) United States
(12) Patent Application Publication
(10) Pub. No.: US 2018/0028594 A1
(43) Pub. Date: Feb. 1, 2018

(54) PERIPHERAL KAPPA OPIOID RECEPTOR AGONISTS FOR HARD TISSUE PAIN

(71) Applicant: Cara Therapeutics, Inc., Stamford, CT (US)

(72) Inventors: Derek T. Chalmers, Riverside, CT (US); James B Jones, Metuchen, NJ (US)

(73) Assignee: Cara Therapeutics, Inc., Stamford, CT (US)

(21) Appl. No.: 15/523,947

(22) PCT Filed: Nov. 3, 2015
(86) PCT No.: PCT/US15/58779

§ 371 Date: May 3, 2017

Related U.S. Application Data
(60) Provisional application No. 62/075,388, filed on Nov. 5, 2014.

(51) Int. Cl. A61K 38/08; A61K 31/485; A61K 31/40

(52) U.S. Cl. CPC A61K 38/08; A61K 31/40; A61K 31/485

(57) ABSTRACT

A method for preventing, inhibiting or treating hard tissue pain in a mammalian subject, the method comprising administering an effective amount of a peripherally-restricted kappa opioid receptor agonist to the subject. The hard tissue pain can be associated with bone, tendons, or cartilage. The peripherally-restricted kappa opioid receptor agonist can be a L-amino acid-containing peptide, a D-amino acid-containing peptide, or a synthetic peptide amide, such as for instance, CR845.

Severity of pain is the key factor in determining an appropriate therapy. Mild or mild-to-moderate pain is generally treated with over the counter products, such as stand-alone oral formulations of aspirin, acetaminophen and ibuprofen. Moderate-to-severe pain, on the other hand, is typically treated with products containing traditional mu opioids. Mu opioid analgesics are effective to some degree for many patients, but have a poor side effect and abuse liability profile, which limits or precludes their use in treating less severe pain. For many people with moderate-to-severe pain, opioid analgesics are the only effective method of treating pain. As a result, these opioid analgesics are among the largest prescription drug classes in the United States. Opioid analgesics represented approximately 71% of the nearly 341 million analgesic prescriptions written in the U.S. in 2012, accounting for an estimated $8.3 billion in sales.

Postoperative pain represents a substantial part of the overall incidence of acute pain. More than 46 million inpatient and 53 million outpatient surgeries are performed annually in the United States. Moderate-to-severe pain in a hospital or other medical setting is most often treated with injectable analgesics. The U.S. intravenous (I.V.) or injectable analgesic therapy market primarily consists of mu opioid agonists, such as morphine, hydromorphone and fentanyl, and certain non-opioid analgesics, such as Toradol (and related generic I.V. ketorolac products), Caldolor (I.V. ibuprofen), and Ofirmev (I.V. acetaminophen).

The standard of care for treating acute postoperative pain, such as bone aches and bone pain is multimodal analgesia, which includes the administration of two or more drugs that act by different mechanisms for providing analgesia in a manner that will minimize the occurrence of adverse events. After hospital treatment, when patients are ready for discharge, a transition is typically made to a prescription oral pain medication, allowing patients to self-administer relatively strong analgesics after being discharged. This transition from an I.V. pain medication to an oral pain medication is referred to as I.V.-to-oral “step-down” therapy.

Strong mu opioid analgesics, such as morphine, fentanyl, and hydromorphone, are mainstays of pain treatment in the immediate postoperative period, and are used as part of a multimodal analgesic approach. However, the use of strong mu opioid analgesics is associated with an array of unwanted and serious side effects, including postoperative opioid-induced respiratory depression, or POIRD, postoperative nausea and vomiting, or PONV, and opioid-induced bowel dysfunction, or OBD, which contributes to the severity of postoperative ileus, or POI. According to Anesthesiology News, the incidence of POIRD may be as high as 29 percent, can occur unexpectedly in even the healthiest of patients, and exerts a disproportionately high toll on length of stay and hospital costs due to the significant expenses associated with the treatment of POIRD. PONV occurs in approximately one-third of surgical patients overall, and is an important factor in determining length of stay after surgery, resulting in annual costs in the U.S. in the range of $1 billion. These mu opioid-related adverse events not only significantly increase the cost of care, but also reduce a patient's quality of care and lead to sub-optimal recovery.

Non-opioid analgesics formulated for injection or infusion, including I.V. acetaminophen and NSAIDs, such as I.V. ibuprofen, are available as alternatives to mu opioids to relieve acute pain, but their use in postoperative care is limited as a result of their lower efficacy. Acetaminophen and NSAIDs also have side effects that limit their use at higher, more efficacious doses. Acetaminophen is associated with risk of liver toxicity, which can be fatal, and NSAIDs are associated with risks of bleeding, serious gastrointestinal side effects including ulcers, kidney damage, and serious thrombotic events such as stroke and heart attack, which can be fatal.

The most common causes of moderate-to-severe chronic pain are musculoskeletal problems and inflammatory conditions. Injuries from accidents resulting in fractures, dislocations or soft tissue injury, as well as lower back pain, are the most frequent causes of musculoskeletal pain, including bone pain. Moderate-to-severe chronic pain is typically treated with prescription products including immediate release and long-acting opioids, such as the branded products Oxycontin (oxycodone) and Opana (oxymorphone), and combination products that include an opioid combined with an NSAID or acetaminophen, such as Vicodin (hydrocodone and acetaminophen) and Percocet (oxycodone and acetaminophen). Prescription products for chronic pain are usually in oral tablet or capsule form because the vast majority of these patients take these medications outside of the hospital setting.

In 2005, the FDA announced a requirement for boxed warnings of potential cardiovascular risk for all NSAIDs. The FDA warning related to cardiovascular adverse events associated with NSAIDs and the increased awareness of the risk of liver toxicity associated with high doses of acetaminophen have led to increased use of mu opioid analgesics for the treatment of chronic pain. However, the use of mu opioid analgesics carries significant additional risks. Chronic opioid use causes patients to develop tolerance for the opioid, which results in the patient needing increasing opioid doses to achieve the same level of pain relief. For the most commonly prescribed analgesic combination products, the need for increasing doses to achieve the same level of pain relief means exposure to increasing amounts of NSAIDs or acetaminophen, which carry the risks attendant to these therapeutics. Moreover, due to their CNS activity, mu opioids produce feelings of euphoria, which can give rise to abuse and addiction. Underlining the severity of this issue, in 2013, the FDA announced class-wide safety labeling changes and new postmarket study requirements for all extended-release and long-acting mu opioid analgesics intended to treat pain. In addition, as a result of their potential for misuse, abuse and addiction, currently approved mu opioids are strictly regulated by the United States Drug Enforcement Agency (DEA), under the Controlled Substances Act, which imposes strict registration, record keeping and reporting requirements, security control and restrictions on prescriptions—all of which significantly increase the costs and the liability attendant to prescription opioid analgesics.

Despite the need for a non-narcotic for pain management, there has been little innovation in the development of new analgesics, with nearly all recent new drug approvals limited being to reformulations and improved methods of delivery of existing therapeutics. Mu opioids continue to be the most prescribed drugs for pain management, despite their side effects and the potential for misuse, abuse and addiction. These concerns often cause health care providers to administer or prescribe less than optimal doses of mu opioids, or patients to take lower than prescribed doses, resulting in inadequate pain relief. Consequently, pain, particularly musculoskeletal and bone pain represents a therapeutic area with substantial unmet need, for physicians who must balance pain control with risks of causing severe adverse events, and for healthcare organizations that bear the costs of managing the consequences of undertreated pain and drug-related adverse events. CR845 therapy, with its novel mechanism of action, presents an improved treatment for moderate-to-severe pain, including hard tissue pain, such as bone pain, because of it provides pain relief without opioid-related adverse events or abuse and addiction issues associated with the currently most commonly used mu opioid analgesics.

### SUMMARY

The present invention provides a method for preventing, inhibiting or treating hard tissue pain in a mammalian subject, the method comprising administering an effective amount of a peripherally-restricted kappa opioid receptor agonist to the subject. In one embodiment, the peripherally-restricted kappa opioid receptor agonist includes a peptide. In another embodiment, the peptide includes one or more D-amino acids.

In one embodiment the present invention provides a method for preventing, inhibiting or treating hard tissue pain in a mammalian subject, the method comprising administering an effective amount of a peripherally-restricted kappa opioid receptor agonist, wherein the peripherally restricted kappa opioid receptor agonist comprises a synthetic peptide amide having the formula:

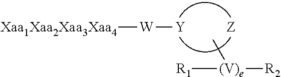

or a stereoisomer, mixture of stereoisomers, prodrug, pharmaceutically acceptable salt, hydrate, solvate, acid salt hydrate, N-oxide or isomorphic crystalline form thereof.

In one embodiment In one embodiment, the residue Xaa₁ is selected from the group consisting of (A)(A′)D-Phe, (A)(A′)(α-Me)D-Phe, D-Tyr, D-Tic, D-tert-leucine, D-neopentylglycine, D-phenylglycine, D-homophenylalanine, and f3-(E)D-Ala, wherein each (A) and each (A′) are phenyl ring substituents independently selected from the group consisting of —H, —F, —Cl, —NO₂, —CH₃, —CF₃, —CN, and —CONH₂, and wherein each (E) is independently selected from the group consisting of cyclobutyl, cyclopentyl, cyclohexyl, pyridyl, thienyl and thiazolyl; Xaa₂ is selected from the group consisting of (A)(A′)D-Phe, 3,4-dichloro-D-Phe, (A)(A′)(α-Me) D-Phe, D-1Nal, D-2Nal, D-Tyr, (E)D-Ala and D-Trp; Xaa₃ is selected from the group consisting of D-Nle, D-Phe, (E)D-Ala, D-Leu, (α-Me)D-Leu, D-Hle, D-Val, and D-Met; Xaa₄ is selected from the group consisting of (B)₂D-Arg, (B)₂D-Nar, (B)₂D-Har, ζ-(B)D-Hlys, D-Dap, ε-(B)D-Lys, ε-(B)₂-D-Lys, D-Amf, amidino-D-Amf, ε-(B)₂D-Dbu, ε-(B)₂α-(B′)D-Orn, D-2-amino-3(4-piperidyl)propionic acid, D-2-amino-3(2-aminopyrrolidyl)propionic acid, D-α-amino-β-amidino propionic acid, α-amino-4-piperidineacetic acid, cis-α,4-diaminocyclohexane acetic acid, trans-α,4-diaminocyclohexaneacetic acid, cis-α-amino-4-methylaminocyclo-hexane acetic acid, trans-α-amino-4-methylaminocyclohexane acetic acid, α-amino-1-amidino-4-piperidineacetic acid, cis-α-amino-4-guanidinocyclohexane acetic acid, and trans-α-amino-4-guanidinocyclohexane acetic acid; wherein each (B) is independently selected from the group consisting of H and C₁-C₄ alkyl, and (B′) is H or (α-Me); W is selected from the group consisting of: Null, provided that when W is null, Y is N; —NH—(CH₂)_{b}— with b equal to zero, 1, 2, 3, 4, 5, or 6; and —NH—(CH₂)_{c}—O— with c equal to 2, or 3, provided that Y is C.

In another embodiment, the moiety

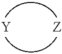

is an optionally substituted 4 to 8-membered heterocyclic ring moiety wherein all ring heteroatoms in said ring moiety are N; wherein Y and Z are each independently C or N; provided that when such ring moiety is a six, seven or eight-membered ring, Y and Z are separated by at least two ring atoms; and provided that when such ring moiety has a single ring heteroatom which is N, then such ring moiety is non-aromatic; V is C₁-C₆ alkyl, and e is zero or 1, wherein when e is zero, then V is null and R₁ and R₂ are directly bonded to the same or different ring atoms; wherein (i) R₁ is selected from the group consisting of —H, —OH, halo, —CF₃, —NH₂, —COOH, C₁-C₆ alkyl, C₁-C₆ alkoxy, amidino, C₁-C₆ alkyl-substituted amidino, aryl, optionally substituted heterocyclyl, Pro-amide, Pro, Gly, Ala, Val, Leu, Ile, Lys, Arg, Orn, Ser, Thr, —CN, —CONH₂, —COR′, —SO₂R′, —CONR′R″, —NHCOR′, OR′ and SO₂NR′R″; wherein said optionally substituted heterocyclyl is optionally singly or doubly substituted with substituents independently selected from the group consisting of C₁-C₆ alkyl, C₁-C₆ alkoxy, oxo, —OH, —Cl, —F, —NH₂, —NO₂, —CN, —COOH, and amidino; wherein R′ and R″ are each independently −H, C₁-C₈ alkyl, aryl, or heterocyclyl or R′ and R″ are combined to form a 4- to 8-membered ring, which ring is optionally singly or doubly substituted with substituents independently selected from the group consisting of C₁-C₆ alkyl, -C₁-C₆ alkoxy, —OH, —Cl, —F, —NH₂, —NO₂, —CN, —COOH and amidino; and R₂ is selected from the group consisting of —H, amidino, singly or doubly C₁-C₆ alkyl-substituted amidino, —CN, —CONH₂, —CONR′R″, —NHCOR′, —SO₂NR′R″ and —COOH; or (ii) R₁ and R₂ taken together can form an optionally substituted 4- to 9-membered heterocyclic monocyclic or bicyclic ring moiety which is bonded to a single ring atom of the Y and Z-containing ring moiety; or (iii) R₁ and R₂ taken together with a single ring atom of the Y and Z-containing ring moiety can form an optionally substituted 4- to 8-membered heterocyclic ring moiety to form a spino structure; or (iv) R₁ and R₂ taken together with two or more adjacent ring atoms of the Y and Z-containing ring moiety can form an optionally substituted 4- to 9-membered heterocyclic monocyclic or bicyclic ring moiety fused to the Y and Z-containing ring moiety; wherein each of said optionally substituted 4-, 5-, 6,-, 7-, 8- and 9-membered heterocyclic ring moieties comprising R₁ and R₂ is optionally singly or doubly substituted with substituents independently selected from the group consisting of C₁-C₆ alkyl, C₁-C₆ alkoxy, optionally substituted phenyl, oxo, —OH, —Cl, —F, —NH₂, —NO₂, —CN, —COOH, and amidino; provided that when the Y and Z-containing ring moiety is a six or seven membered ring having a single ring heteroatom and e is zero, then R₁ is not —OH, and R₁ and R₂ are not both —H; and provided further that when the Y and Z-containing ring moiety is a six membered ring having two ring heteroatoms, both Y and Z are N and W is null, then —(V)ₑR₁R₂ is attached to a ring atom other than Z; and if e is zero, then R₁ and R₂ are not both —H.

### BRIEF DESCRIPTION OF THE FIGURES

FIG. 1: Efficacy of CR845 in “Chung Model” of neuropathic pain is blocked with Peripheral (Intrapaw) administration of a kappa antagonist (norBNI) in rats. *** denotes p<0.001. compared to vehicle-treated controls (two-way ANOVA). Vehicle or Nor-BNI was administered intraplantarly (0.2 mg) 15 min prior to CR845. Injection (1 mg/kg). N=6 male rats/group, mean±SEM.

FIG. 2: Phase 2b Laparoscopic Hysterectomy—Summed Pain Intensity Difference from 0-24 Hours (SPID₀₋₂₄) following postoperative treatment. *p≦0.05, **p≦0.01.

FIG. 3: Phase 2b Laparoscopic Hysterectomy—Pain Intensity Difference (PID) at specific times relative to postoperative baseline pain intensity. *p≦0.05, **p≦0.01 for CR845/CR845. #p≦0.05 for both Placebo/CR845 and CR845/Placebo. Values represent mean±SEM.

FIG. 4: Phase 2b Laparoscopic Hysterectomy—Total Pain Relief Within the first 2 hours (TOTPAR0-2) following postoperative treatment. *p<0.05. Values represent mean±SEM.

FIG. 5: Phase 2b Laparoscopic Hysterectomy—Morphine Consumption For 2-24 hours post-treatment in patients. *p≦0.05; Values represent mean±SEM.

FIG. 6: Phase 2b Laparoscopic Hysterectomy—Incidence of opioid-related adverse events over 24 hours. ***p≦0.001; *p<0.05.

FIG. 7: Phase 2b Laparoscopic Hysterectomy—Responder analysis of global evaluation of study medication. **p=0.001.

FIG. 8a: Phase 2 Bunionectomy—Summed Pain Intensity Difference from 0-24 hours (SPID₀₋₂₄), 0-36 hours (p SPIDO-36) and 0-48 hours (SPIDO-48) in completer population.

FIG. 8b: Phase 2 Bunionectomy—Summed Pain Intensity Difference from 0-24 hours (SPIDO-24), 0-36 hours (SPIDO-36) and 0-48 hours (SPIDO-48) in mITT Population (Completers plus non-completers). *p≦0.05—One-sided ANOVA with Treatment Group as a Main Effect (mean +/−SEM).

FIG. 9a: Phase 2 Bunionectomy—Pain Intensity Difference relative to baseline in CR845 and placebo completer treatment groups over a 48 hour period. * p≦0.05 (0-36 hours). ** p≦0.01 (0-12 hours).

FIG. 9b: Phase 2 Bunionectomy—Pain Intensity Difference relative to baseline in CR845 and placebo treatment Groups in mITT populations across 48 hours. *p≦0.05 (0-12 hours).

FIG. 10: Phase 2 Bunionectomy—CR845 Suppression of Nausea and Vomiting. *p<0.05.

### DETAILED DESCRIPTION

Kappa opioid receptor agonists and their uses for the prophylaxis, inhibition and treatment of diseases, disorders and conditions of soft tissues are described in U.S. Pat. Nos. 7,402,564; 7,713,937; 7,727,963; 7,842,662; 8,217,007; 8,486,894; and 8,536,131, the disclosures of which are hereby incorporated by reference herein in their entireties.

In one embodiment the present invention provides a method for preventing, inhibiting or treating hard tissue pain in a mammalian subject such as a human, the method comprising administering an effective amount of a peripherally-restricted kappa opioid receptor agonist to the subject, wherein the moiety:

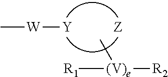

is selected from the group consisting of:

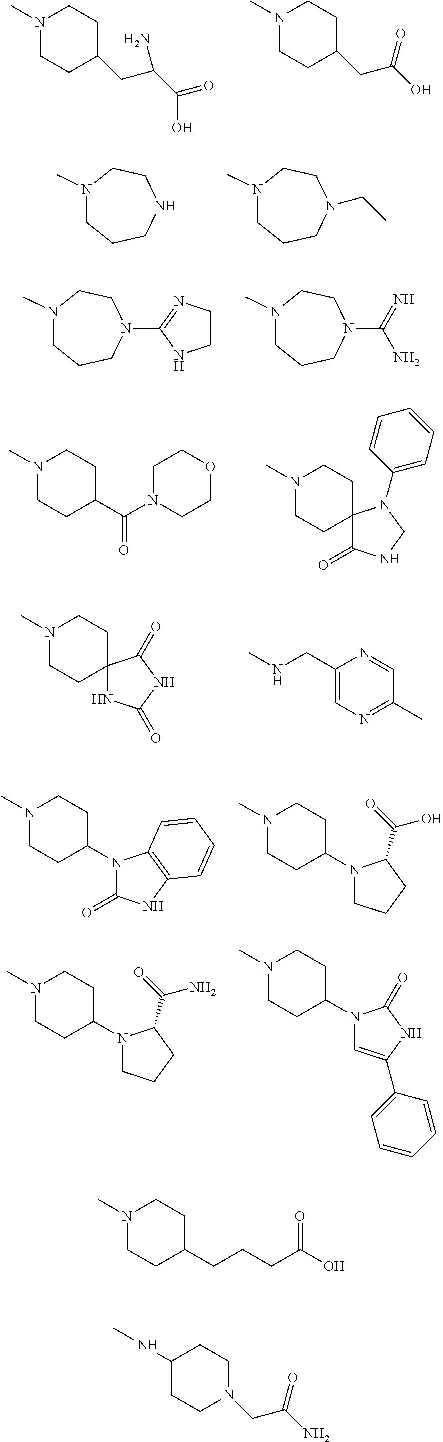

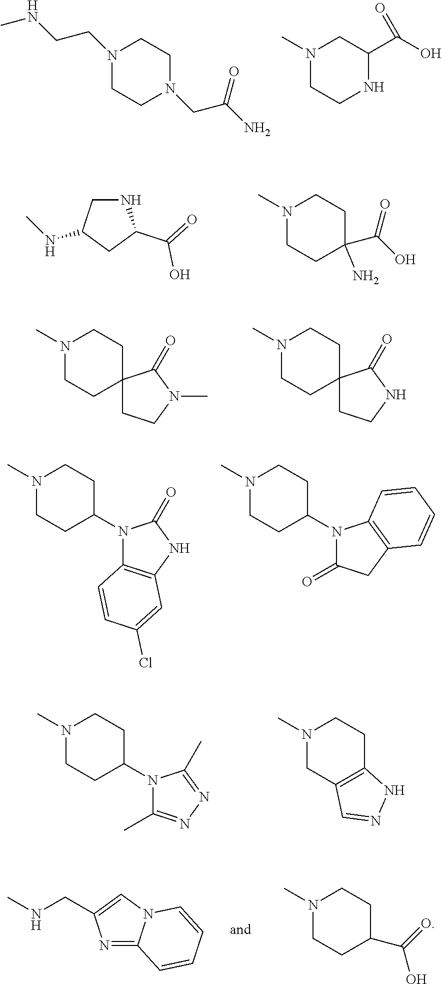

In another embodiment, the invention provides a method for preventing, inhibiting or treating hard tissue pain in a mammalian subject, the method comprising administering an effective amount of a peripherally-restricted kappa opioid receptor agonist to the subject, wherein the synthetic peptide amide has the structure:

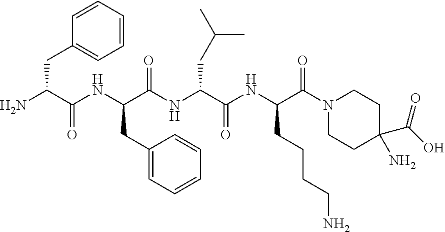 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]

D-Phe-D-Phe-D-Leu-D-Lys-[ω(4-aminopiperidine-4-carboxylic acid)]—OH (also called CR845). The peripherally-restricted kappa opioid receptor agonist can be administered to the subject within 12, 24 or 36 hours prior to, during or within 12, 24 or 36 hours after undergoing a medical procedure. In one embodiment, the medical procedure causes hard tissue pain, e.g. bone pain.

In another embodiment, the invention provides a method for preventing, inhibiting or treating hard tissue pain in a mammalian subject, wherein the peripherally-restricted kappa opioid receptor agonist is administered to the subject after a physical insult such as an abrasion, a cut, a bone fracture, and an open wound. In another embodiment, the peripherally-restricted kappa opioid receptor agonist is administered by a route of injection selected from the group consisting of subcutaneous injection, intravenous injection, intraperitoneal injection, intra-articular injection, and intramuscular injection.

In another embodiment, the peripherally-restricted kappa opioid receptor agonist can be any suitable peripherally-restricted kappa opioid receptor agonist, such as for instance a non-narcotic analgesic, for example, asimadoline (N-[(1S)-2-[(3S)-3-hydroxypyrrolidin-1-yl]-1-phenylethyl]-N-methyl-2,2-diphenylacetamide), or nalfurafine ((2E)-N-[(5α,6β)-17-(cyclo-propylmethyl)-3,14-dihydroxy-4,5-epoxymorphinan-6-yl]-3-(3-furyl)-N-methylacrylamide).

Hard tissue is defined as a tissue having a rigid intercellular substance. An example of a hard tissue is bone. In humans, hard tissues include bones, cartilages and teeth. Skeletal bones and cartilages are examples of hard tissues in mammals. Mineralized tissues combine stiffness, low weight, strength and toughness due to the presence of minerals in soft protein networks and tissues. Approximately sixty different minerals are generated through biological processes; the most common ones are calcium carbonate found in mollusk shells and hydroxyapatite present in teeth and bones. Studies have shown that mineralized tissues are 1,000 to 10,000 times tougher than the minerals they contain due to the organized layering of the tissue. As a consequence of his layering, loads and stresses are transferred through several length-scales, from macro to micro to nano, which results in the dissipation of energy within the arrangement.

Bone is a mineralized tissue with a hierarchical structure that is also formed by the self-assembly of smaller components. The mineral in bone is hydroxyapatite that also includes carbonate ions, while the organic portion is made mostly of collagen and other proteins. Hydroxyapatite, also called hydroxylapatite (HA), is a naturally occurring mineral form of calcium apatite with the formula Ca₅(PO₄)₃OH), but is usually written Ca₁₀(PO₄)₆(OH)₂ to denote that the crystal unit cell includes two entities. Hydroxyapatite crystallizes in the hexagonal crystal system. Pure hydroxylapatite powder is white. Naturally occurring apatites can, however, also have brown, yellow, or green colorations, comparable to the discolorations of dental fluorosis. Bone mineral includes up to 50% by volume and 7% by weight of a modified form of hydroxyapatite. Dental enamel and dentin are composed mainly of carbonated calcium-deficient hydroxyapatite. Hydroxyapatite crystals are also found in the small calcifications (within the pineal gland and other structures) known as corpora arenacea a.k.a. “brain sand.”

Bone is a complex biological material. The hierarchical structures of bone are divided into macroscale, microscale and nanoscale structures. The macroscale structures, from several millimetres to centimeters are visible as compact bone and spongy bone. The microscale bone structures include two hierarchical structures. First, from 100 μm to 1 mm, inside the compact bone where cylindrical units called osteons and small struts can be distinguished. The second hierarchical structure, the ultrastructure, at a scale of 5 to 10 μm, is the actual structure of the osteons and small struts, On the nanoscale, there are also two hierarchical structures: The first is the structure inside the ultrastructure of the fibrils and extrafibrillar space, at a scale of several hundred nanometres. The second nanoscale structure includes the elementary components of mineralized tissues at a scale of tens of nanometres. The components of this nanoscale structure are the mineral crystals of hydroxyapatite, cylindrical collagen molecules, organic molecules such as lipids and proteins, and finally water. Mineral is the inorganic component of mineralized tissues. This constituent is what makes the tissues harder and stiffer. Hydroxyapatite, calcium carbonate, silica, calcium oxalate, and monosodium urate are examples of minerals found in biological tissues. In bone, studies have shown that calcium phosphate nucleates within the lumen of the collagen fibrils and then grows in these zones until it occupies the entire space.

The organic component of mineralized tissues, such as bone is made up of proteins. In bone, the organic layer is the protein collagen. The degree of mineral in mineralized tissues varies and the organic component occupies a smaller volume as tissue hardness increases. However, without this organic portion, the biological material would be brittle and fragile. Many proteins are regulators of the mineralization process. They act in the nucleation or inhibition of hydroxyapatite formation. Some of the regulatory proteins in mineralized tissues are osteonectin, osteopontin, osteocalcin, bone sialoprotein and dentin phosphophoryn.

Hard tissue pain is one of the most severe forms of pain and is often managed with mu opioids. However, such long term treatment of chronic hard tissue pain suffers from the opioid-related adverse events or abuse and addiction issues associated with the currently most commonly used mu opioid analgesics. Until the introduction of the peripherally-restricted synthetic peptide amide compounds, such as CR845, the previously tested kappa opioids shared the adverse effects of the mu opioids. The present invention provides a novel and surprisingly efficacious therapy for hard tissue pain, including bone pain.

Bone pain is a debilitating form of pain emanating from the bone tissue. Bone pain can be due to a wide range of diseases or physical conditions and may severely impair the quality of life for patients who suffer from it. Bone pain belongs to the class of deep somatic pain, often experienced as a dull pain that cannot be localized accurately by the patient. This is in contrast with the pain which is mediated by superficial receptors such as those in the skin. Bone pain can have several possible causes ranging from extensive physical stress to serious diseases such as cancer. For many years it has been known that bones are innervated with sensory neurons. More recently, it is becoming clear what types of nerves innervated which sections of bone. The periosteal layer of bone tissue is highly pain-sensitive and an important cause of pain in several disease conditions causing bone pain, like fractures, osteoarthritis, etc. In certain diseases the endosteal and haversian nerve supply seems to play an important role, e.g., in osteomalacia, osteonecrosis, and other bone diseases.

### Kappa Receptor Agonist CR845

CR845 is a peripherally-acting kappa opioid receptor agonist useful for treatment of both acute and chronic pain. The most advanced product candidate, I.V. CR845, has demonstrated significant pain relief and a favorable safety and tolerability profile in three Phase 2 clinical trials in patients with acute postoperative pain. Due to its selectivity for the kappa opioid receptor and ability to decrease mu opioid use, CR845 has demonstrated a consistent ability to decrease the acute opioid-related adverse events (AEs) of nausea and vomiting with no evidence of drug-related respiratory depression. CR845 has been administered to over 300 human subjects in Phase 1 and Phase 2 clinical trials as an intravenous infusion, short bolus or oral capsule and was considered to be safe and well tolerated in these clinical trials.

CR845-based products, when approved, will be attractive for patients with moderate-to-severe pain and their physicians due to the following attributes:
Its novel, peripherally-acting, kappa opioid receptor mechanism of action;
Strong evidence of its efficacy;
The reduction of mu opioid use and opioid-related AEs, such as nausea and vomiting;
The avoidance of mu opioid-related CNS side effects, such as respiratory depression and euphoria;
The absence of euphoria which lowers addiction or abuse potential;
The avoidance of drug-drug interactions; and
Its availability in I.V. form for acute pain treatment in the hospital setting and oral form for treatment of acute and chronic pain in either a hospital or outpatient setting.

In standard preclinical pain models, CR845 successfully attenuated acute and chronic visceral, inflammatory and neuropathic pain in a dose-dependent manner (see Table 1, below). The analgesic effect of CR845 was recordable within 15 minutes post-administration and lasted for up to 18 hours following single-dose administration. CR845 also decreased the production and release of pro-inflammatory mediators, likely due to the direct activation of kappa opioid receptors expressed on immune cells that synthesize and secrete these substances.

TABLE 1
CR845 Exhibits a Broad Spectrum of Activity in Multiple
Types of Standard Preclinical Pain Models

| | | | ED₅₀ | Duration of | |
|---|---|---|---|---|---|
| Model | | Species | (I.V., mg/kg) | Action | |
| Somato - Visceral | Acetic Acid Writhing - | Mouse | 0.07 | >18 | hours |
| Inflammatory | Somatic and visceral pain | | | | |
| Pain | | | | | |
| Chronic | Complete Freund's Adjuvant - | Rat | 0.08 | >2 | hours |
| Inflammatory | mechanical hyperalgesia | | | | |
| Pain | | | | | |
| Acute | Carrageenan - mechanical | Rat | 0.3 | >1 | hour |
| Inflammatory | hyperalgesia | | | | |
| Pain | | | | | |
| Neuropathic | L5/6 Spinal Nerve Ligation - | Rat | 0.3 | >8 | hours |
| Pain | tactile allodynia | | | | |

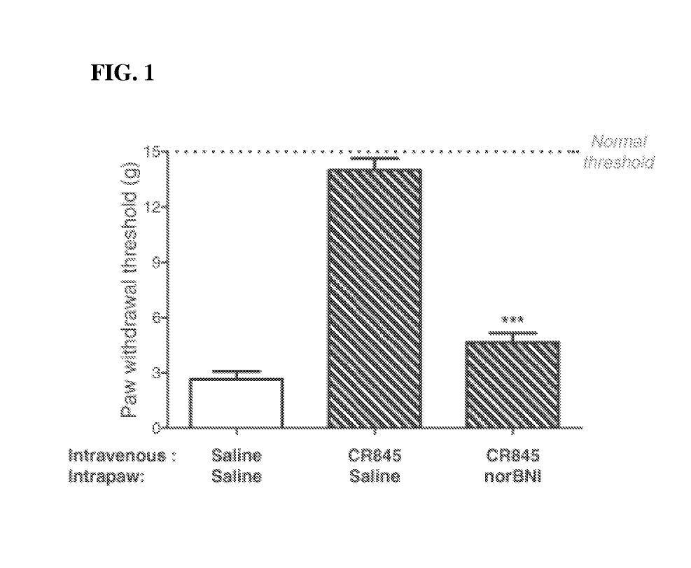

The peripheral mechanism of action of CR845 is supported preclinically by both biochemical in vitro assays and in vivo functional pharmacological studies. In pharmacokinetic studies, animals administered analgesic and supra-analgesic doses of CR845 exhibited no measurable concentrations of drug in extracted brain tissue indicating that the CNS was not the site of action for CR845. Moreover, in standard preclinical pain models, such as the “Chung Model” of neuropathic pain, the analgesic action of CR845 was blocked with kappa opioid receptor antagonists administered directly to the local site of injury, indicating a peripheral site of action for CR845 (FIG. 1). In the “Chung Model”, neuropathic pain is induced experimentally by ligating spinal nerves mediating sensation for a hind limb. This results in a type of neuropathic pain, referred to as allodynia. Experimental animals with allodynia exhibit a “paw withdrawal reflex” upon contact with a relatively thin filament on the injured site. Sets of different thickness filaments are used to test sensitivity, each of which is designed to produce a given force (in grams) upon bending after contact. By testing with these filaments, the minimum force to evoke a withdrawal response defines the paw withdrawal threshold. The nerve injury produces a marked reduction in paw withdrawal thresholds (increased sensitivity to force) in response to probing with the filaments. I.V. administration of CR845 reduces this neuropathic pain as demonstrated by a subsequent increase in the withdrawal threshold (see FIG. 1).

Administration of a low dose of the selective peripherally-acting kappa opioid receptor antagonist nor-binaltorphamine, or nor-BNI, into the plantar surface of the injured paw significantly reduces the effect of CR845, whereas injection of saline had no effect on the efficacy of CR845. Since nor-BNI was only able to block local peripheral kappa opioid receptors in this experiment, these results show that the effect of CR845 is a result of activation of kappa opioid receptors located at the peripheral site of injury rather than in the CNS.

Intravenous CR845

CR845, in an injectable version of the most advanced kappa opioid receptor-based peripheral analgesic is designed to provide pain relief without stimulating mu opioid receptors and therefore without mu opioid-related side effects, such as nausea, vomiting, respiratory depression and euphoria. Intravenous CR845 has demonstrated efficacy and tolerability in three randomized, double-blind, placebo-controlled Phase 2 clinical trials in patients undergoing soft tissue (laparoscopic hysterectomy) and hard tissue (bunionectomy) surgery. In both the laparoscopic hysterectomy and bunionectomy clinical trials, CR845 administration resulted in statistically significant reductions in pain intensity, as measured by summed pain intensity differences, or SPID, which is the FDA-recommended acute pain endpoint.

A Phase 2 clinical trial (CLIN2002) was a multicenter, double-randomized, double-blind, placebo-controlled trial conducted in 203 patients at 22 sites in the United States. The trial enrolled female patients, ages 21 to 65, scheduled for elective laparoscopic hysterectomy under general anesthesia. In this trial, patients were administered either placebo or one dose of 0.04 mg/kg I.V. CR845 preoperatively. Following surgery, if they were medically stable and had a pain intensity score 40 on a 100 point pain scale based on the visual analog scale, or VAS, they were re-randomized to receive either placebo or one dose of 0.04 mg/kg I.V. CR845. Efficacy was measured using time-specific 24 hour pain intensity differences. Pain intensity, or PI, was measured at various times by asking patients to rate their pain on a 100-point scale, where “0” is absence of pain and “100” is the worst possible pain. PID, or pain intensity difference, is the difference between the PI measured prior to treatment and at subsequent times of measurement. SPID, or the summed pain intensity difference, is the time-weighted sum of all of the PID scores, from the pretreatment level to a subsequent time of measurement, such as 24 hours after the pretreatment baseline pain measurement. Both PID and SPID are FDA-recognized endpoints for acute pain clinical trials. Additional endpoints included the amount of morphine consumption over 24 hours, time-specific total pain relief and patient global evaluation of study medication. Of the 203 patients that participated in the trial, 183 received a post operative dose; however, two subjects did not record baseline pain scores and were not included in calculated PID and SPID values. Accordingly, four treatment groups resulted from preoperative and postoperative randomization:
(1) I.V. CR845 administered both preoperatively and postoperatively (CR845/CR845);
(2) placebo administered preoperatively and I.V. CR845 administered postoperatively (Placebo/CR845);
(3) I.V. CR845 administered preoperatively and placebo administered postoperatively (CR845/Placebo); and
(4) placebo administered both preoperatively and postoperatively (Placebo/Placebo).

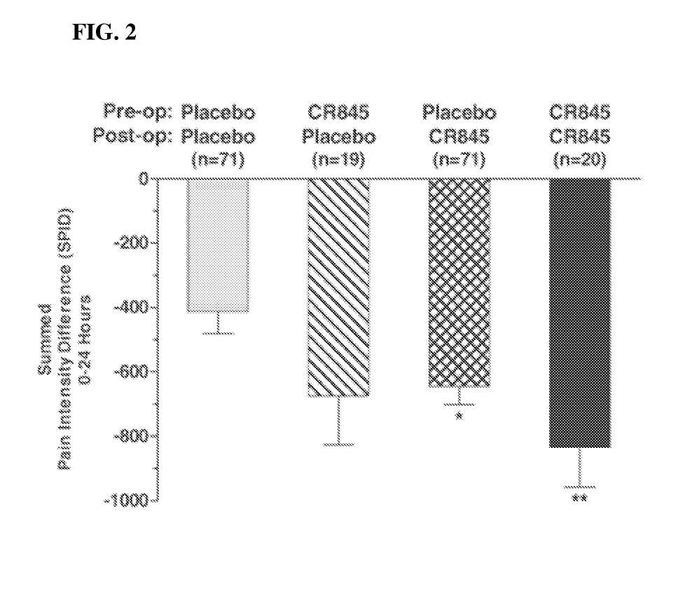

The CR845/CR845 group exhibited a statistically significant reduction in pain over a 24-hour time period, as indicated by an improvement in 0-24 hour mean SPID, compared to the Placebo/Placebo group (p≦0.01). The Placebo/CR845 group also exhibited a statistically significant improvement in 0-24 hour mean SPID compared to the Placebo/Placebo group (p≦0.05). The CR845/Placebo group exhibited an improved 0-24 hour mean SPID compared to the Placebo/Placebo group, but this difference did not reach statistical significance, which we believe was due to the small number of patients. FIG. 2 illustrates the 0-24 hour mean SPIDs of the four treatment groups listed above.

Similar observations were made for different time periods after treatment. For example, over the 0-4 hour time period, in the CR845/CR845 group, there was a statistically significant 3.5-fold improvement in mean SPID values compared to the Placebo/Placebo group (p≦0.05). In addition, over the 0-8, 0-12 and 0-16 time periods, patients in the Placebo/CR845 group also exhibited reduced pain intensity compared to the Placebo/Placebo group in a statistically significant manner (p≦0.05), based on improved SPID values.

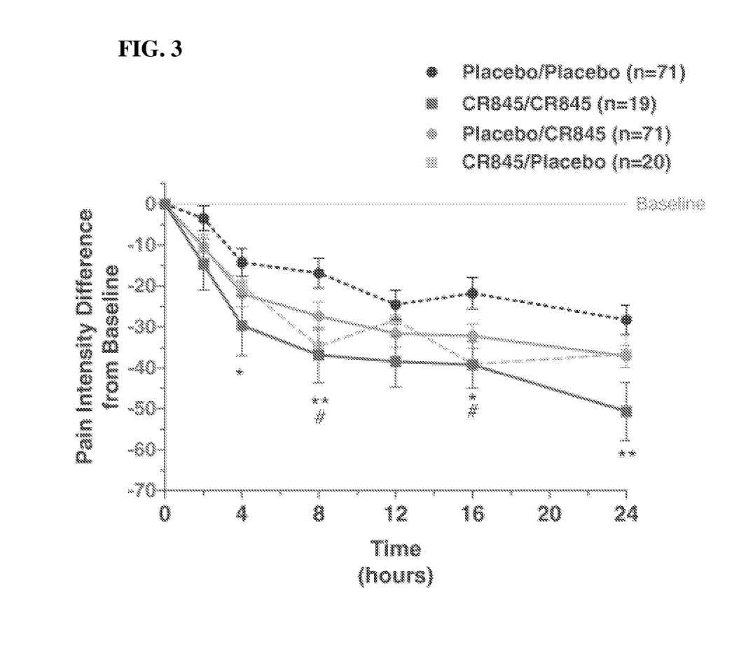

The mean PID from baseline at each time interval was numerically superior across all groups that received I.V. CR845 preoperatively and/or postoperatively relative to the Placebo/Placebo group. Compared to the Placebo/Placebo group, patients in the CR845/CR845 group exhibited an approximately 60% greater reduction in pain intensity at 24 hours (p≦0.01), as well as statistically significant improvements for the 0-4, 0-8 and 0-16 hour time intervals. Patients in the CR845/Placebo and Placebo/CR845 groups also exhibited statistically significant decreases in pain intensity for the 0-8 and 0-16 hour time intervals, compared to patients in the Placebo/Placebo group. FIG. 3 illustrates the PID relative to postoperative baseline in patients in the four treatment groups.

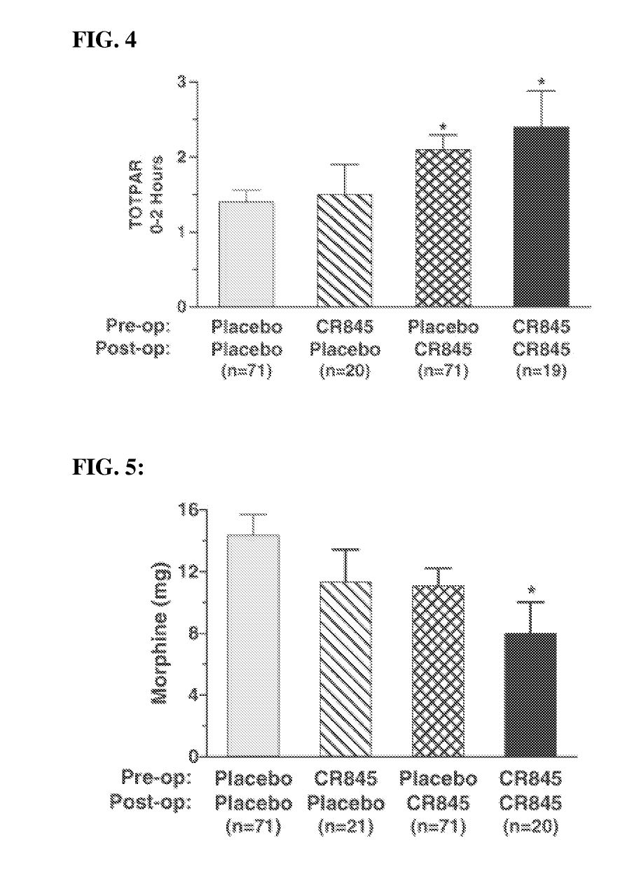

At the same time points at which pain intensity measurements were taken, patients' perceived pain relief scores were recorded using a 5 point subjective Likert scale (0-4), where zero corresponds to no relief and a score of four represents total relief. The “TOTPAR” score is calculated as the “total pain relief score”, which is a time-weighted sum of pain relief scores over any given time period following post operative treatment with CR845 or placebo. Mean TOTPAR scores were numerically superior across all intervals for the CR845/CR845 and Placebo/CR845 groups relative to the Placebo/Placebo group. The patients in the CR845/CR845 group and Placebo/CR845 exhibited statistically superior pain relief as compared to the Placebo/Placebo group within the first 2 hours following postoperative randomization, as indicated by increased mean TOTPAR₀₋₂ values (p≦0.05). FIG. 4 depicts the mean TOTPAR scores for the first 2 hour period for each of the four treatment groups listed above.

In the CR845/CR845 and Placebo/CR845 groups, there were also statistically significant improvements in reported pain relief for the 0-4, 2-4 and 0-8 hour time periods. In addition, the improvement in mean TOTPAR also reached statistical significance for the 0-12 hour interval for the CR845/CR845 group relative to the Placebo/Placebo group.

Intravenous morphine was available as rescue medication to all treatment groups upon patient request. Calculations of morphine consumption per treatment group in the 2-24 hour period, after patients leave the post-anesthesia care unit, or PACU, indicated that patients in the CR845/CR845 group used approximately 45% less morphine than those in the Placebo/Placebo group (p≦0.05) and patients in the Placebo/CR845 and CR845/Placebo groups used approximately 23% less morphine than those in the Placebo/Placebo group. FIG. 5 depicts the morphine usage in each of the treatment groups between hours 2-24.

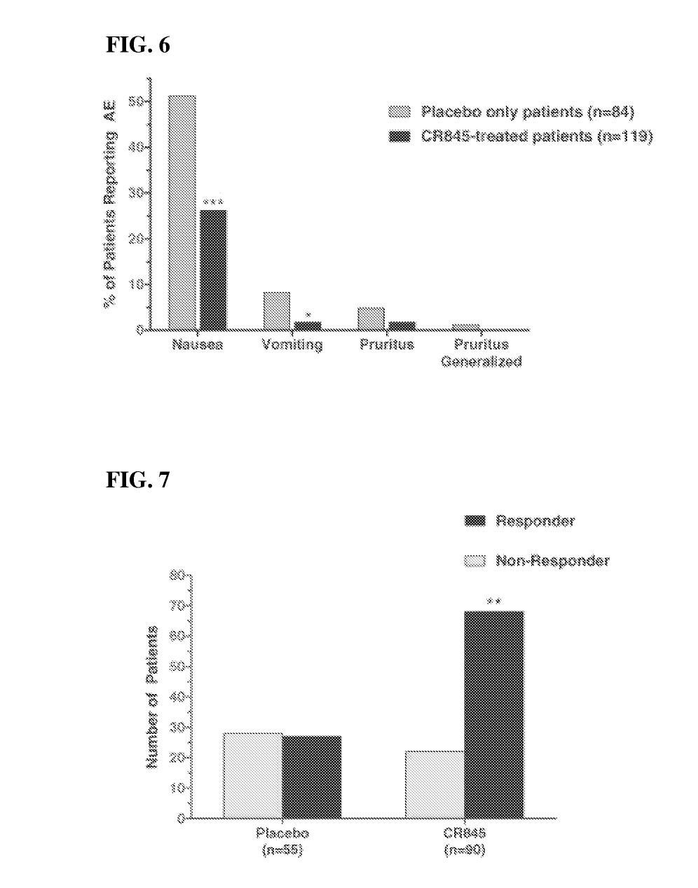

Concurrently with the observed reduction in morphine use, patients treated with I.V. CR845 exhibited a statistically significant lower incidence of opioid-related AEs through 24 hours after the start of the first infusion compared to patients who received only placebo. The incidence of nausea was reduced by approximately 50% (only 26.1% of patients administered CR845 experienced nausea as compared to 51.2% for placebo, p≦0.001) and the incidence of vomiting was reduced nearly 80% (only 1.7% of patients administered CR845 experienced vomiting, as compared to 8.3% for placebo, p=0.035). There was also less pruritus, or itching sensation, reported in patients treated with CR845 compared to placebo. FIG. 6 depicts the percentage of patients reporting opioid-related adverse events of nausea, vomiting and pruritus.

In addition to the reduction of opioid-related adverse events, a standard responder analysis indicated that a higher percentage of patients who received I.V. CR845 were characterized as “Responders” as compared to those receiving placebo (p=0.001). Responders included patients who rated their medication “Excellent” or “Very Good” and Non-Responders as those who rated their medication “Fair” or “Poor”. The lower overall pain intensity scores at the end of the study period for CR845-treated patients and the significant reduction in nausea and vomiting reported in these patients contributed to patients' greater satisfaction with I.V. CR845 treatment compared to placebo. FIG. 7 depicts the number of patients classified as Responders or Non-Responders in the I.V. CR845-treated patients compared to the patients receiving only placebo.

In this trial, intravenous administration of 0.04 mg/kg of I.V. CR845 preoperatively and/or postoperatively was safe and generally well tolerated. The placebo and CR845 treatment patient groups showed a similar overall incidence of treatment-emergent adverse events, or TEAEs, the majority of which were mild to moderate in severity. The most frequent TEAEs, reported in 10% or more of total patients, were nausea, hypotension, flatulence, blood sodium increase, or hypernatremia, and headache. There were no apparent consistent differences between CR845 and placebo groups in clinical laboratory results, vital signs, electrocardiogram, or oxygen saturation results, with the exception of blood sodium increase, which was evident only in CR845 treatment groups (14% of total patients). The increase in blood sodium levels, or hypernatremia, observed in CR845 treatment groups was likely a result of the aquaretic effect of I.V. CR845 at this dose and the replacement of fluid loss with sodium-containing intravenous solutions, rather than water or low to no sodium-containing fluids. In subsequent trials, fluid replacement with water or I.V. solutions with low or no sodium were used and no evidence of hypernatremia was observed.

### CR845 for Bunionectomy

Bunionectomy is a surgical procedure to remove a bunion, which is an enlargement of the joint at the base of the big toe and includes bone and soft tissue. The procedure typically results in intense pain requiring significant postoperative analgesic care, usually beginning with local anesthetic infusion and ongoing administration of a strong opioid, such as morphine or fentanyl, for several days after surgery.

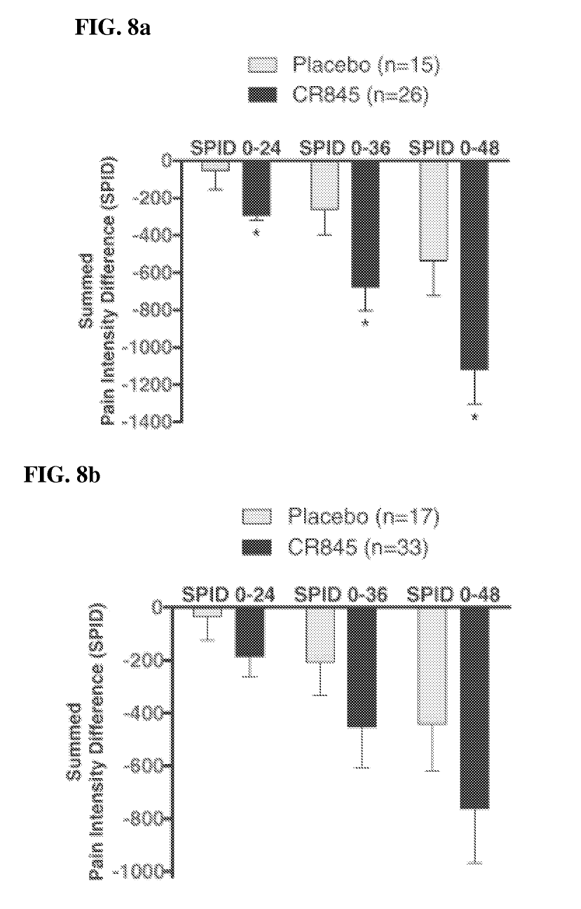

Clinical trial (CLIN2003) was a randomized, double-blind, placebo-controlled trial conducted in 51 patients following bunionectomy surgery at a single site in the U.S. The trial enrolled female and male patients, ages 18 years and older, scheduled for elective bunionectomy under regional anesthesia. Using a standard clinical trial protocol in which local anesthetic infusion was terminated on the day after surgery, patients were randomized into one of two treatment groups (CR845 or Placebo, in a 2:1 ratio) after reporting moderate-to-severe pain, defined as a pain intensity score ÷40 on a 100-point pain scale. Patients randomized to receive I.V. CR845 were administered an I.V. injection at a dose of 0.005 mg/kg, and additional doses on an as-needed basis 30-60 minutes later, and then no more frequently than every 8 hours through a 48-hour dosing period. The results were analyzed separately for the per protocol population, or “Completers”, which includes only patients who completed the trial, and the modified Intent-to-Treat, or mITT, population, which includes Completers and all patients who discontinued the trial, or “non-Completers”. In the Completer group, CR845 treatment resulted in a statistically significant reduction in pain intensity compared to placebo, as measured by the SPID score over the initial 24 hour time period (SPID₀₋₂₄; p<0.05). This reduction in pain intensity after CR845 dosing was also statistically significant over a 36 hour time period (SPID₀₋₃₆, p<0.03), as well as over the entire two-day dosing period (SPID₀₋₄₈, p<0.03), compared to placebo-treated patients (see FIG. 8a). Numerical improvements in SPID scores in the CR845 group as compared to placebo were also evident across the same time periods when analyzing the mITT population of Completers together with non-Completers (see FIG. 8b).

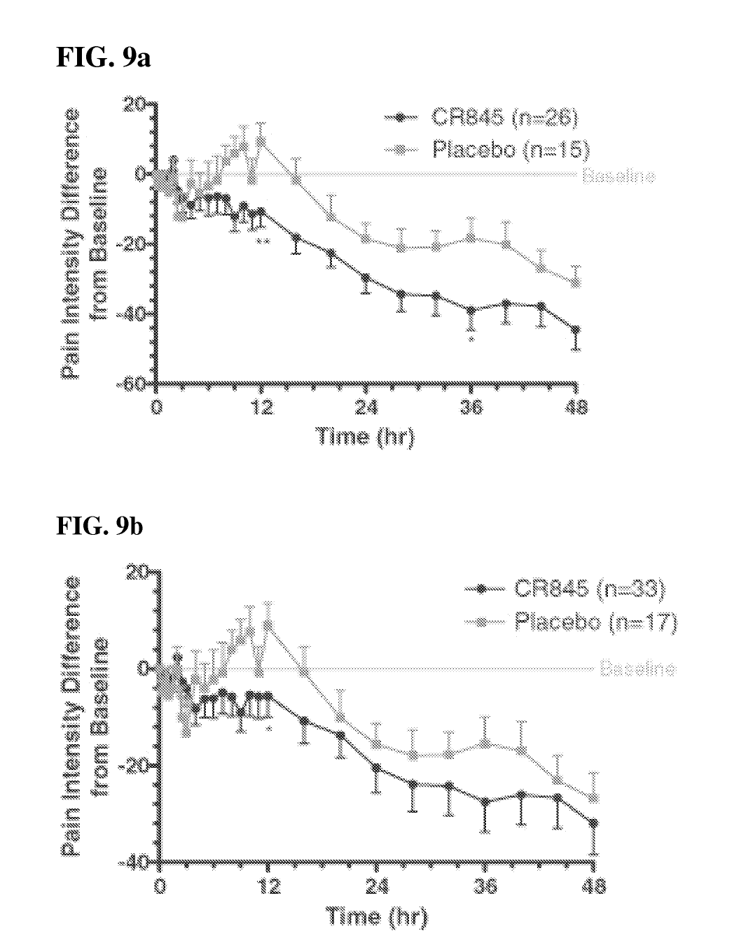

The Completer analysis is indicative of the actual efficacy of I.V. CR845 under conditions where patients are exposed to the drug as specified in the protocol, while the mITT analysis is indicative of the actual variability that will be encountered in the mITT populations. The understanding of this variability serves as the basis for determining the appropriate number of patients for enrollment in our Phase 3 clinical trials. In this trial, mean PID from baseline at each time interval was measured, and was numerically superior across the 48 hour trial period in the I.V. CR845 treatment group relative to the placebo group for both the Completer and mITT populations (see FIGS. 9a and 9b). Statistically significant reductions in pain intensity differences in the CR845 group versus placebo were evident in the 0-12 hour time interval for both the Completer and mITT populations (p≦0.01 and p≦0.05 respectively) and for the 0-36 hour time interval for the Completer populations (p≦0.05), consistent with the findings with the primary SPID endpoints.

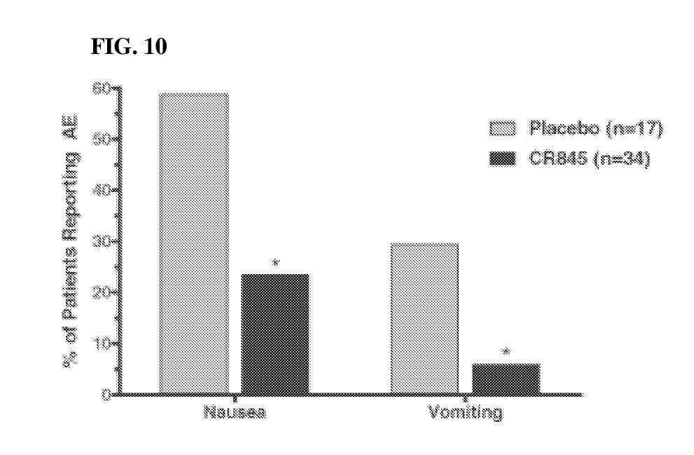

Fentanyl was available to both CR845 and placebo treatment groups upon patient request. While there was no difference in mean fentanyl use between the placebo and CR845 groups, the incidence of opioid-related AEs of nausea and vomiting was significantly reduced (by 60% and 80%, respectively; p≦0.05) in patients who received CR845 compared to placebo during the 48 hour period after randomization (see FIG. 10).

The ability of I.V. CR845 to reduce nausea and vomiting despite not meaningfully reducing fentanyl usage is believed to be due to a direct antiemetic effect resulting from its kappa opioid agonist mechanism of action. The ability to provide postsurgical analgesia and simultaneously reduce opioid-related side effects makes I.V. CR845 an attractive treatment option for postoperative patients and their physicians.

In this bunionectomy trial, repeated intravenous administration of I.V. CR845 at a dose of 0.005 mg/kg was safe and generally well tolerated. The most frequent TEAEs (greater than 10%) observed in the CR845 treatment group were transient facial tingling and somnolence. Of the seven cases of somnolence reported, four were reported as “mild” and/or “related to drug” and three as “moderate” and/or “not related to drug”. The mean plasma sodium concentration in CR845-treated patients exhibited an approximately 3% rise over 24 hours from baseline levels, but was not outside the normal physiological range at either 24 or 48 hours post-CR845 administration. This lack of clinically significant hypernatremia was likely a result of both utilizing a lower dose of I.V. CR845 and replacing transient fluid loss with oral water or sodium-free intravenous fluid. In addition, consistent with our prior studies, there was no evidence of acute psychiatric side effects that were observed with prior-generation CNS-active kappa opioid agonists.

### CR845 Phase 1 Clinical Trials

In addition to the three Phase 2 clinical trials, the safety of CR845 has been demonstrated in four Phase 1 clinical trials. CR845 was generally well tolerated in all of these clinical trials. The most common TEAEs across evaluated populations were transient facial tingling or numbness, dizziness, fatigue and a transient increase in urine output in the absence of electrolyte loss, or aquaresis. Some of the subjects with aquaresis also exhibited an increase in heart rate upon standing up, or postural tachycardia, which was not accompanied by a decrease in blood pressure, resolved without intervention, and was classified as mild by the Investigator. This elevation in heart rate was demonstrated to be a physiological consequence of the subject's fluid deficit rather than a direct effect of the drug. No other changes in vital signs, including supine pulse rate, blood pressure, respiratory rate, oral body temperature, or oxygen saturation were reported, nor were any clinically significant changes observed in electrocardiogram characteristics. Additionally, the CNS adverse events characteristic of prior-generation CNS-active kappa agonists, such as acute psychiatric side effects, were not observed with CR845. The potential to cause sedation was assessed using the Ramsey Sedation Scale in the ascending dose-tolerance Phase 1 trial (Study 2048-001) of I.V. CR845, which included 54 subjects (17 on placebo; 37 on CR845). CR845 did not cause sedation in this population of normal, healthy subjects in this trial.

### I.V. CR845 For Acute Pain

I.V. CR845 for the management of acute postoperative pain in adult patients: The market for management of postoperative pain is highly fragmented and can be segmented into three general classes of products:
mu opioid-based products, such as morphine, fentanyl, hydrocodone, and hydromorphone, all of which are available generically;
local anesthetic-based products, such as lidocaine and bupivacaine, which are available generically; and
adjunctive analgesics, which are defined as non-mu opioid pain-relieving drugs that provide additional control of postoperative pain.

There has been a trend in recent years for anesthesiologists to a use all three classes of products to manage postoperative pain, often referred to as “multimodal analgesia.” When approved, I.V. CR845 will be competing within the overall acute postoperative pain market, although it is expected that it would compete primarily with adjunctive analgesics, particularly in multimodal analgesic treatment approaches. Common adjunctive analgesics include: ketorolac, an injectable NSAID, which is available generically; Caldolor, an injectable; and Ofirmev, an injectable acetomenophen.

1. A method for preventing, inhibiting or treating hard tissue pain in a mammalian subject, the method comprising administering an effective amount of a peripherally-restricted kappa opioid receptor agonist to the subject.

2. The method according to claim 1, wherein the peripherally-restricted kappa opioid receptor agonist comprises a peptide.

3. The method according to claim 1, wherein the peripherally-restricted kappa opioid receptor agonist comprises one or more D-amino acids.

4. The method according to claim 1, wherein the peripherally restricted kappa opioid receptor agonist comprises a synthetic peptide amide having the formula: 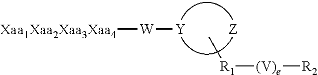
or a stereoisomer, mixture of stereoisomers, prodrug, pharmaceutically acceptable salt, hydrate, solvate, acid salt hydrate, N-oxide or isomorphic crystalline form thereof; wherein
Xaa₁ is selected from the group consisting of (A)(A′)D-Phe, (A)(A′)(α-Me)D-Phe, D-Tyr, D-Tic, D-tert-leucine, D-neopentylglycine, D-phenylglycine, D-homophenylalanine, and β-(E)D-Ala, wherein each (A) and each (A′) are phenyl ring substituents independently selected from the group consisting of —H, —F, —Cl, —NO₂, —CH₃, —CF₃, —CN, and —CONH₂, and wherein each (E) is independently selected from the group consisting of cyclobutyl, cyclopentyl, cyclohexyl, pyridyl, thienyl and thiazolyl;
Xaa₂ is selected from the group consisting of (A)(A′)D-Phe, 3,4-dichloro-D-Phe, (A)(A′)(α-Me)D-Phe, D-1Nal, D-2Nal, D-Tyr, (E)D-Ala and D-Trp;
Xaa₃ is selected from the group consisting of D-Nle, D-Phe, (E)D-Ala, D-Leu, (α-Me)D-Leu, D-Hle, D-Val, and D-Met;
Xaa₄ is selected from the group consisting of (B)₂D-Arg, (B)₂D-Nar, (B)₂D-Har, ζ-(B)D-Hlys, D-Dap, ε-(B)D-Lys, ε-(B)₂-D-Lys, D-Amf, amidino-D-Amf, γ-(B)₂D-Dbu, δ-(B)₂α-(B′)D-Orn, D-2-amino-3(4-piperidyl)propionic acid, D-2-amino-3 (2-aminopyrrolidyl)propionic acid, D-α-amino-β-amidinopropionic acid, α-amino-4-piperidineacetic acid, cis-a,4-diaminocyclohexane acetic acid, trans-α,4-diaminocyclohexaneacetic acid, cis-α-amino-4-methylaminocyclo-hexane acetic acid, trans-α-amino-4-methylaminocyclohexane acetic acid, α-amino-1-amidino-4-piperidineacetic acid, cis-α-amino-4-guanidinocyclohexane acetic acid, and trans-α-amino-4-guanidinocyclohexane acetic acid, wherein each (B) is independently selected from the group consisting of H and C₁-C₄ alkyl, and (B′) is H or (α-Me);
W is selected from the group consisting of:
Null, provided that when W is null, Y is N;
—NH—(CH₂)_{b}— with b equal to zero, 1, 2, 3, 4, 5, or 6; and
—NH—(CH₂)_{c}—O— with c equal to 2, or 3, provided that Y is C; the moiety 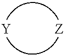
is an optionally substituted 4 to 8-membered heterocyclic ring moiety wherein all ring heteroatoms in said ring moiety are N; wherein Y and Z are each independently C or N;
provided that when such ring moiety is a six, seven or eight-membered ring, Y and Z are separated by at least two ring atoms; and provided that when such ring moiety has a single ring heteroatom which is N, then such ring moiety is non-aromatic;
V is C₁-C₆ alkyl, and e is zero or 1, wherein when e is zero, then V is null and R₁ and R₂ are directly bonded to the same or different ring atoms;
wherein (i) R₁ is selected from the group consisting of —H, —OH, halo, —CF₃, —NH₂, —COOH, C₁-C₆ alkyl, C₁-C₆ alkoxy, amidino, C₁-C₆ alkyl-substituted amidino, aryl, optionally substituted heterocyclyl, Pro-amide, Pro, Gly, Ala, Val, Leu, Ile, Lys, Arg, Orn, Ser, Thr, —CN, —CONH₂, —COR′, —SO₂R′, —CONR′R″, —NHCOR′, OR′ and SO₂NR′R″; wherein said optionally substituted heterocyclyl is optionally singly or doubly substituted with substituents independently selected from the group consisting of C₁-C₆ alkyl, C₁-C₆ alkoxy, oxo, —OH, —Cl, —F, —NH₂, —NO₂, —CN, —COOH, and amidino; wherein R′ and R″ are each independently —H, Cᵢ-C₈ alkyl, aryl, or heterocyclyl or R′ and R″ are combined to form a 4- to 8-membered ring, which ring is optionally singly or doubly substituted with substituents independently selected from the group consisting of C₁-C₆ alkyl, -C₁-C₆ alkoxy, —OH, —Cl, —F, —NH₂, —NO₂, —CN, —OOH and amidino; and R₂ is selected from the group consisting of —H, amidino, singly or doubly C₁-C₆ alkyl-substituted amidino, —CN, —CONH₂, —CONR′R″, —NHCOR′, —SO₂NR′R″ and —COOH; or
(ii) R₁ and R₂ taken together can form an optionally substituted 4- to 9-membered heterocyclic monocyclic or bicyclic ring moiety which is bonded to a single ring atom of the Y and Z-containing ring moiety; or
(iii) R₁ and R₂ taken together with a single ring atom of the Y and Z-containing ring moiety can form an optionally substituted 4- to 8-membered heterocyclic ring moiety to form a spino structure; or
(iv) R₁ and R₂ taken together with two or more adjacent ring atoms of the Y and Z-containing ring moiety can form an optionally substituted 4- to 9-membered heterocyclic monocyclic or bicyclic ring moiety fused to the Y and Z-containing ring moiety;
wherein each of said optionally substituted 4-, 5-, 6,-, 7-, 8- and 9-membered heterocyclic ring moieties comprising R₁ and R₂ is optionally singly or doubly substituted with substituents independently selected from the group consisting of C₁-C₆ alkyl, C₁-C₆ alkoxy, optionally substituted phenyl, oxo, —OH, —Cl, —F, —NH₂, —NO₂, —CN, —COOH, and amidino;
provided that when the Y and Z-containing ring moiety is a six or seven membered ring having a single ring heteroatom and e is zero, then R₁ is not —OH, and R₁ and R₂ are not both —H; and
provided further that when the Y and Z-containing ring moiety is a six membered ring having two ring heteroatoms, both Y and Z are N and W is null, then —(V)ₑR₁R₂ is attached to a ring atom other than Z; and if e is zero, then R₁ and R₂ are not both —H.

5. The method of claim 4, wherein the moiety: 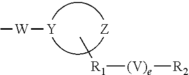
is selected from the group consisting of:

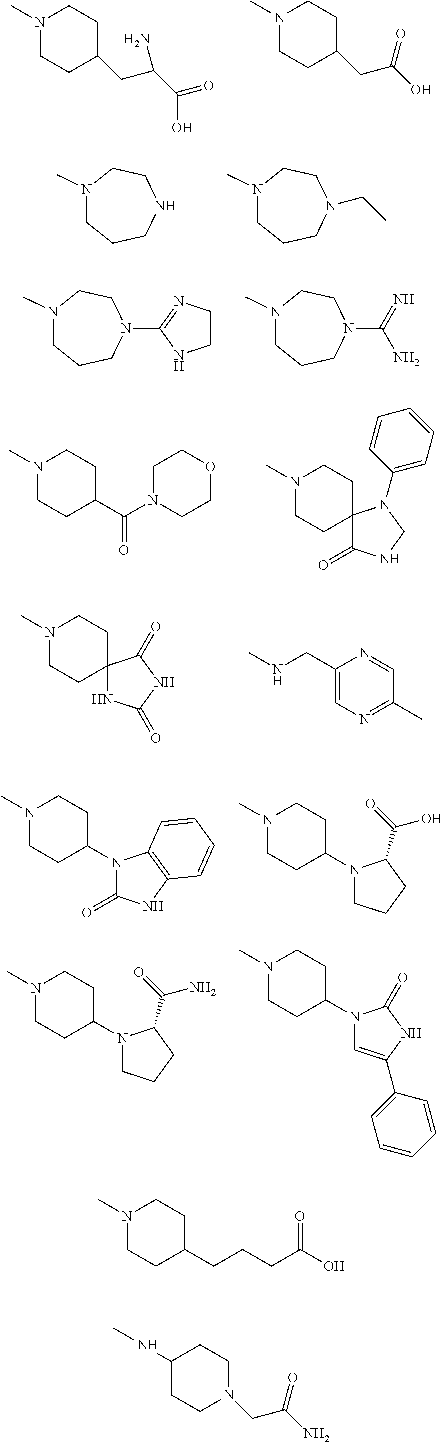

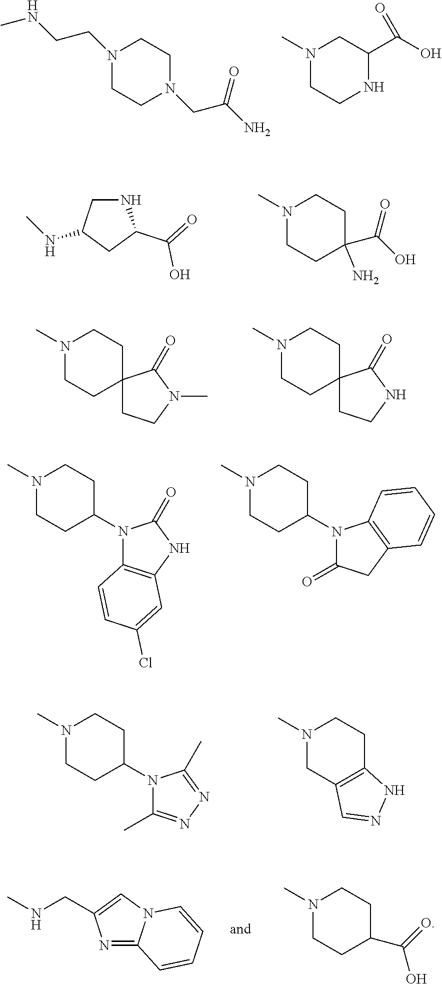

6. The method of claim 4, wherein the synthetic peptide amide has the structure: 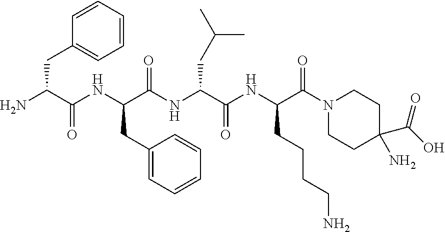 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]
D-Phe-D-Phe-D-Leu-D-Lys-[ω(4-aminopiperidine-4-carboxylic acid)]—OH.

7. The method of claim 6, wherein the mammalian subject is a human.

8. The method according to claim 1, wherein the peripherally-restricted kappa opioid receptor agonist is administered to the subject within 24 hours prior to, during, or within 24 hours after undergoing a medical procedure.

9. The method according to claim 8, wherein the medical procedure causes bone pain.

10. The method according to claim 6, wherein the peripherally-restricted kappa opioid receptor agonist is administered to the subject after a physical insult selected from the group consisting of an abrasion, a cut, a bone fracture, and an open wound.

11. The method according to claim 1, wherein the peripherally-restricted kappa opioid receptor agonist is administered by a route of injection selected from the group consisting of subcutaneous injection, intravenous injection, intraperitoneal injection, intra-articular injection, and intramuscular injection.

12. The method according to claim 1, wherein the peripherally-restricted kappa opioid receptor agonist is a non-narcotic analgesic.

13. The method according to claim 1, wherein the peripherally-restricted kappa opioid receptor agonist is asimadoline (N-[(1S)-2-[(3S)-3-hydroxypyrrolidin-1-yl]-1-phenylethyl]-N-methyl-2,2-diphenylacetamide).

14. The method according to claim 1, wherein the peripherally-restricted kappa opioid receptor agonist is nalfurafine ((2E)-N-[(5α,6β)-17-(cyclopropylmethyl)-3,14-dihydroxy-4,5-epoxymorphinan-6-yl]-3-(3-furyl)-N-methylacrylamide).

15. The method according to claim 1, wherein the hard tissue comprises bone, cartilage, or a combination of bone and cartilage.

16. The method according to claim 15, wherein the mammal is a human.

17. The method according to claim 9, wherein the mammal is a human.

18. The method according to claim 17, wherein the medical procedure is bunionectomy.

19. The method according to claim 18, wherein the peripherally-restricted kappa opioid receptor agonist is administered by a route of injection selected from the group consisting of subcutaneous injection, intravenous injection, intraperitoneal injection, intra-articular injection, and intramuscular injection.

20. The method according to claim 19, wherein the peripherally-restricted kappa opioid receptor agonist is administered by intravenous injection.
